(19) United States
(12) Patent Application Publication
(10) Pub. No.: US 2009/0131980 A1
(43) Pub. Date: May 21, 2009

(54) Tendon Cap and method for tendon repair

(76) Inventors: Irvin M. Wiesman, Hinsdale, IL (US); Brian J. Bear, Rockford, IL (US)

Correspondence Address: KNECHTEL, DEMEUR & SAMLAN, 525 W. MONROE STREET, SUITE 2360, CHICAGO, IL, 60661, US

(21) Appl. No.: 12/313,568

(22) Filed: Nov. 20, 2008

Related U.S. Application Data
(60) Provisional application No. 60/989,156, filed on Nov. 20, 2007.

(51) Int. Cl. A61B 17/04; A61B 19/00

(52) U.S. Cl. 606/228; 606/232; 128/898; 606/148

(57) ABSTRACT

A method and apparatus for joining opposite ends of a severed tendon or ligament. A core suture is attached to one of the severed ends. A cap is attached to the severed end by the core suture. The cap protects the severed end from trauma and fraying as it is pulled back toward the opposite severed end of the tendon. The cap is removed prior to reattachment of the severed ends. The cap may have a coating applied to it to reduce friction and add other properties such as improved healing to the tendon after it is reattached.

### RELATED APPLICATION

This application claims the priority of U.S. Provisional Application No. 60/989,156 filed Nov. 20, 2007.

### BACKGROUND AND SUMMARY OF THE INVENTION

When a tendon is severed, the proximal tendon end retracts similar to a cut rubber band. A surgeon must reconnect the tendon by guiding at least one severed end through the fibrous/osseous tunnels comprising the pulley system along the bones in the hand and fingers to re-attach the severed proximal and distal ends. The tendon must be safely pulled through the passageways so that minimal tearing or fraying of the ends occurs. Passing lacerated or divided tendons underneath pulley systems and through fibro osseous and osseous tunnels can often result in tendon injury, tendon end fraying and traumatic injury to the tendon epitenon. This has been related to increased tendon adhesion formation, increased bulk at repair sites, decreased repair strength, and increased risk of tendon rupture and loss of tendon excursion upon final healing.

In the inventive procedure, once the tendon is pulled through the pulleys, the ends of the tendons are normally secured together by sutures tied to the opposed ends of the tendons in a conventional surgical procedure. Upon re-attachment, the body will undergo a normal healing process to repair the severed tendon.

A major problem found in this procedure is that the ends of the tendons can fray as they are being pulled back through the pulleys. Prior passing techniques normally utilize sutures or a grasper that manage to pull the tendon through the pulleys along the bone but cause tearing or fraying of the tendon in the process. The fraying can cause damage to the tendon and increase the amount of time necessary to allow the tendons to heal once they have been re-connected. In order to achieve a more successful tendon repair surgery, both ends of the tendons should be free from frayed ends. The surgical procedure also takes more time to complete if there are frayed ends on the tendon, which makes the reattachment more difficult. This can increase the risk of complications.

Recent advances in tendon research have demonstrated that certain growth factors, mesemchymal cell, mucopolysaccharides, organic and inorganic lubricants, and virus mediated gene transfer of growth factors can promote tendon healing and/or decrease adhesion formation after tendon repair. Thus it is an object of this invention to use a method and apparatus that promotes healing by applying one or more of the above mentioned healing substances.

### DESCRIPTION OF THE INVENTION

The present invention proposes a solution to these problems by providing a tendon cap which is simple for a surgeon to use. The present invention greatly reduces fraying by encompassing the entire end of the tendon in elastic material. The tendon cap can be made of a solid or elastic synthetic or non-synthetic material that encompasses the end of the tendon as described herein.

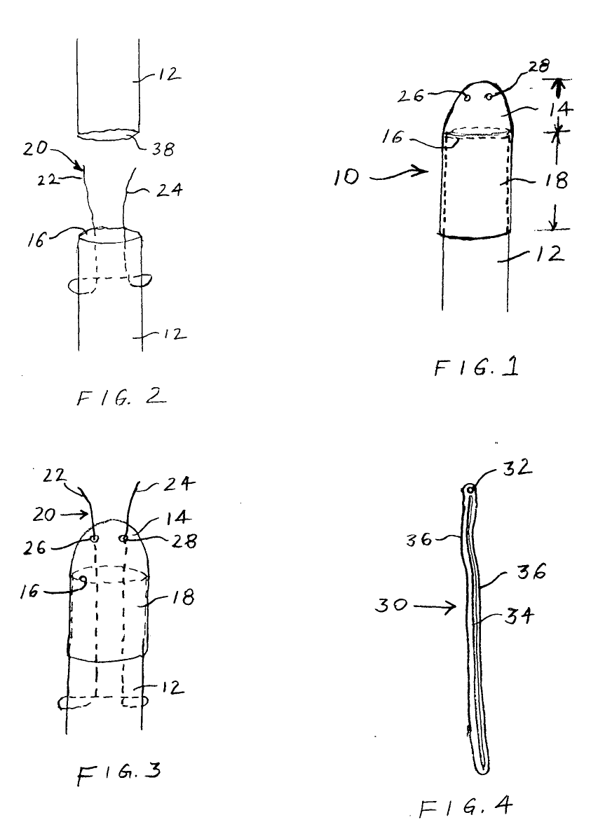

As seen in FIGS. 1 and 2, a tendon cap 10 is formed from a thin membrane tube with a flexible membrane cap that is placed over an end 16 of a lacerated or divided tendon 12. The lacerated tendon 12 has an opposite end 38 to which end 16 is to be re-attached. There are two zones to the tendon cap 10. There is a head 14 that fits directly over the end 16 of the tendon 12 and a body 18 which shrouds the tendon 12 for some length from the end 16. This protective tendon cap 10 is designed for minimizing friction and injury when passing the tendon 12 under pulley systems or through a fibro osseous/osseous tunnel. The tendon 12 will be delivered to the surgical repair site with much less injury to the tendon end 16 and epitenon. This allows for less bulky repair, increased repair strength and decrease in adhesion formation. Upon final healing, tendon strength and excursion will be maximized. The tendon cap 10 can be made of an elastic synthetic or non-synthetic material that encompasses the end 16 of the tendon 12.

The tendon cap 10 can also be coated with specific substances to: (1) decrease friction when passing the tendon 12 with the tendon cap 10 beneath pulleys or through fibro osseous/osseous tunnels; (2) decrease trauma to the tendon end 16 and epitenon when passing the tendon 12 beneath pulleys and through the fibro osseous/osseous tunnels; (3) promote stronger more rapid healing at the repair site; and (4) decrease adhesion formation between the tendon and the surrounding tissue.

### Tendon Cap Inside Surface Coatings

The inside area of the head 14 is the membrane portion of the tendon cap 10 that is in intimate contact with the tendon end 16. The inside of the head 14 can be coated with various substances which will be transferred to the tendon end 16 during the tendon passing process by direct contact with the tendon end 16. These substances are utilized for their ability to promote rapid strong healing at the repair site and to prevent adhesion formation between the tendon 12 and the surrounding tissue. The substances that may be placed on the inside of the tendon cap include but are not limited to: (1) all growth factors that will promote tendon healing and decrease tendon adhesion formation; (2) Platelet derived growth factors; (3) Fibroblast derived growth factors; (4) Mesemchymal stem cells; (5) Virus mediated gene transfer vectors to promote expression of genes for formation of growth factors that promote tendon healing and decrease tendon adhesions; (6) Virus mediated transfer of basic fibroblast growth factor (bFGF) gene through adenosine associated viral-2 (AAV2); (7) growth/differentiation factor 5 (GdF5), recombinant growth/differentiation factor 5 (rhGdF5); and (8) Fibronectin.

### Head Outside Surface Coatings

The outside area of the head 14 is the membrane portion of the head that is in contact with the surrounding tissue through which the tendon 12 is being passed. The outside of the head 14 can be coated with various substances that will be transferred to the tendon and surrounding tissue by direct contact to decrease: (1) friction when passing the tendon 12 with the tendon cap 10 beneath pulleys or through fibro osseous/osseous tunnels; (2) trauma to the tendon end 16 and epitenon when passing the tendon 12 beneath pulleys and through the fibro osseous/osseous tunnels; and (3) adhesion formation between the tendon 12 and the surrounding tissue.

Substances that can be utilized to coat the outside of the tendon cap 10 include but are not limited to: (1) growth/differentiation factor 5 (GdF5), recombinant growth/differentiation factor 5 (rhGdF5); (2) Phospholipids; (3) Lipid derivatives; (4) Muccopolysaccharided; (5) Synthetic or organic lubricants; (6) Virus mediated gene transfer vectors to promote expression of genes for formation of growth factors that promote tendon healing and decrease tendon adhesions; (7) Virus mediated transfer of basic fibroblast growth factor (bFGF) gene through adenosine associated viral-2 (AAV2); and (8) Mesencymal stem cells.

### Body Outside and Inside Coatings

The body 18 on both the outside membrane and inside membrane of the tendon cap 10 can be coated with various substances that can be transferred to the tendon 12 epitenon and surrounding tissue by direct contact to decrease: (1) friction when passing the tendon 12 with the tendon cap 10 beneath pulleys or through fibro osseous/osseous tunnels; (2) trauma to the tendon end 16 and epitenon when passing the tendon 12 beneath pulleys and through the fibro osseous/osseous tunnels; and (3) adhesion formation between the tendon 12 and the surrounding tissue.

Substances that can be utilized to coat the outside of the tendon cap 10 include but are not limited to: (1) growth/differentiation factor 5 (GdF5), recombinant growth/differentiation factor 5 (rhGdF5); (2) Phospholipids; (3) Lipid derivatives; (4) Muccopolysaccharided; (5) Synthetic or organic lubricants; (6) Virus mediated gene transfer vectors to promote expression of genes for formation of growth factors that promote tendon healing and decrease tendon adhesions; (7) Virus mediated transfer of basic fibroblast growth factor (bFGF) gene through adenosine associated viral-2 (AAV2); and (8) Mesencymal stem cells.

### Technique for Passing Cut Flexor Tendons Beneath Pulleys and Through Fibro Osseous Tunnels/Osseous Tunnels

As seen in FIG. 3, a core grasping suture 20 is placed into the cut tendon end 16, exists one side of the tendon 12, passes diametrically back through the tendon 12 and exits the other side of the tendon 12. It then reenters the tendon 12 at the same side through which it just exited and goes back up the central core of the tendon 12 and exits through the cut end 16. Alternatively, any core suture can be utilized as long as there are two to 6 six strands of suture that exit the tendon end 16 to be passed through the tendon cap 10. As seen in FIG. 3, the suture 20 has two free ends 22, 24 which are passed through tendon cap suture target markings 26, 28 in the head 14 in an “inside to outside” fashion. The tendon is then pulled into and secured to the tendon cap 10 utilizing the core sutures 20 as a traction device. Alternatively, the suture 20 can be initially passed through target marking 26, through the end 16 and the tendon 12, back through the end 16 and the other target marking 28. Jeweler's forceps are utilized to help guide the tendon cap 10 over the tendon end 16. The core sutures 20 are then shuttled beneath the pulley system or through the fibro osseous tunnel utilizing a tendon cap suture passing tool 30 such as the one illustrated in FIG. 4. There is a hole 32 at one end of the tool 30 through which the core sutures 20 pass. A malleable wire 34 passes through the center of the tool 30. A synthetic plastic coating 36 covers the malleable wire 34 and encapsulates the entire tool 30 so that it can be manufactured as a prepackaged sterile tool that can be discarded after use. Alternatively it can be made of materials that allow it to be sterilized and re-used.

As the sutures 20 are withdrawn beneath the pulley or through the fibro osseous tunnel, the tendon 12 with the tendon cap 10 in place is gently pulled and passes beneath the pulley system or through the fibro osseous tunnel. The tendon 12 with the tendon cap 10 in place is initially guided beneath the pulley system or into the fibro osseous tunnel utilizing forceps without teeth. During this process, the substances coated on the tendon cap head 14 are transferred onto the tendon end 16 by direct contact. The substances that are placed on the inside and outside of the tendon cap body 18 are transferred onto the tendon epitenon and surrounding pulley system and fibro osseous tunnel by direct contact.

Once the end 16 of the severed tendon is pulled a sufficient distance to be re-attached to the distal end 38 of the tendon 12, the tendon cap 10 can be removed from the tendon 12 and easily slid off the suture 20. The two ends 16, 38 of the tendon 12 can be re-connected through conventional medical procedures such as placing the core suture 20 through the distal end 38. Additional sutures can be used to re-connect the two ends 16, 38 of tendon 12 as necessary or desired by the surgeon or a free or French eye needle can be used with the already placed core suture 20.

By fully encompassing the end 16 of the tendon 12 with the tendon cap 10, tendon fraying is greatly reduced and the patient will enjoy a shorter time to achieve a complete recovery.

Thus there has been provided a method and apparatus for tendon repair that fully satisfies the objects set forth herein. While the invention has been described in conjunction with a specific embodiment, it is evident that many alternatives, modifications and variations will be apparent to those skilled in the art in light of the foregoing description. Accordingly, it is intended to embrace all such alternatives, modifications and variations as fall within the spirit and scope of the appended claims.

What is claimed is:

1. A method for joining opposite severed ends of a tendon comprising the steps of:
a. attaching a core suture to one severed end of a tendon,
b. covering the one severed end of the tendon with a cap having an inside and outside surface, for protecting the one severed end,
c. passing the core suture through the cap for securing the cap to the one severed end,
d. pulling the core suture with the attached tendon and cap toward the other severed end of the tendon,
e. removing the cap, and
f. attaching the opposite severed ends.

2. The method of claim 1 and the further step of attaching the core suture to the severed end of the tendon by passing the core suture through the severed end, through the epitenon, and back through the severed end.

3. The method of claim 1 and the further step of applying a surface coating to the inside of the cap which contacts the severed end.

4. The method of claim 3 and further selecting the surface coating from the group consisting of (1) all growth factors that will promote tendon healing and decrease tendon adhesion formation, (2) Platelet derived growth factors, (3) Fibroblast derived growth factors, (4) Mesemchymal stem cells, (5) Virus mediated gene transfer vectors to promote expression of genes for formation of growth factors that promote tendon healing and decrease tendon adhesions, (6) Virus mediated transfer of basic fibroblast growth factor (bFGF) gene through adenosine associated viral-2 (AAV2), (7) growth/differentiation factor 5 (GdF5), recombinant growth/differentiation factor 5 (rhGdF5), and (8) Fibronectin.

5. The method of claim 1 and the further step of applying an outside surface coating to the outside of the cap.

6. The method of claim 5 and further selecting the outside surface coating from the group consisting of (1) growth/differentiation factor 5 (GdF5), recombinant growth/differentiation factor 5 (rhGdF5), (2) Phospholipids, (3) Lipid derivatives, (4) Muccopolysaccharided, (5) Synthetic or organic lubricants, (6) Virus mediated gene transfer vectors to promote expression of genes for formation of growth factors that promote tendon healing and decrease tendon adhesions, (7) Virus mediated transfer of basic fibroblast growth factor (bFGF) gene through adenosine associated viral-2 (AAV2), and (8) Mesencymal stem cells.

7. The method of claim 1 and the further step of passing a second core suture through the cap for securing the cap to the one severed end.

8. The method of claim 1 and further comprising the step of passing the core suture through tendon cap suture target markings in the cap.

9. The method of claim 1 and further comprising the step of attaching the core suture to a tool for pulling the suture toward the other severed end of the tendon.

10. The method of claim 1 and the further step of axially implanting the core suture in the one severed end of the tendon.

11. The method of claim 1 and the further step of pulling the core suture and tendon cap through the fibrous/osseous tunnels comprising a pulley system along the bones in a person's hand and fingers.

12. A system for aiding in the reattachment of opposite severed ends of a tendon or ligament comprising:
a. a cap having an inside and outside surface, for protecting one severed end of the tendon or ligament,
b. a core suture attached to the one severed end of the tendon or ligament, the core suture passing through the cap for securing the cap to the one severed end,
c. a coating placed on the cap.

13. The system of claim 12 wherein the coating placed on the cap is a friction reducing coating.

14. The system of claim 12 wherein the coating placed on the cap is an adhesion reducing coating to reduce the possibility of adhesion between the tendon or ligament and surrounding tissue.

15. The system of claim 14 and further comprising a tool for receiving the core suture and for pulling the core suture and severed end toward the opposite severed end for reattachment.

16. The system of claim 15 wherein the tool comprises an elongated flexible body with suture retaining means at one end of the tool to hold the core suture as it is pulled toward the opposite severed end.

17. The system of claim 12 wherein the coating is selected from the group consisting of (1) growth/differentiation factor 5 (GdF5), recombinant growth/differentiation factor 5 (rhGdF5), (2) Phospholipids, (3) Lipid derivatives, (4) Muccopolysaccharided, (5) Synthetic or organic lubricants, (6) Virus mediated gene transfer vectors to promote expression of genes for formation of growth factors that promote tendon healing and decrease tendon adhesions, (7) Virus mediated transfer of basic fibroblast growth factor (bFGF) gene through adenosine associated viral-2 (AAV2), and (8) Mesencymal stem cells.
